SEQUENCE LISTING (4 sequences)

SEQ ID NO: 1 - DNA, 818 bp, puccinia graminis
atgaacttcgttcttcaactcagtgttctgctgctcacaatcatctcacagtcttttgttcgatccagtcccttcacaacaagggcaactgaagttatacccgatcctgcaaccacctgctcccggcccggccttgcggcacttacttacgatgacggaccttatgattatgaaaataaaatctccgattacctcaatgcccgtcatattaaaggcaccttctatgtcaacggcaataactacgattgcatttacgatgaggcgatagtgaagcacttaaagcgcactttctcccagggccacttgattggatcacatacatggtcacatgcaaatatcagcagcttatcggccgcagaattaaaccaacagcttgacttagtcgaagtagcattaatcaagattcttggagtcaagccgaaattcttccgacctccttatggtgcattcgaccagaagagcttggcgatattgaagaaacgggggtacatcgtggccaactggtcatttgactccgaagacgctgtgggagcgacgcccgagcaatcgatggcatcttataaggaactctcgaagcagtttccggcctctcagatcacactcaaccatgagacttaccaaactacggccgagaaagtgaccccatatgccgtgcctctgttgcaaaaggccggatataagctagtgcatatatccgaatgcttgggaacgggcaccaaattgactgatctctaccaatgggtcggaaagccttctgtcagagatgcatcgtggacgtgcaatgggaccccagctccaacggacaactaagaattccg

SEQ ID NO: 2 - DNA, 1205 bp, puccinia graminis
atgaacttcgttcttcaactcagtgttctgctgctcacaatcatctcacagtcttttgttcgatccagtcccttcacaacaagggcaactgaagttatacccgatcctgcaaccacctgctcccggcccggccttgcggcacttacttacgatgacggaccttatgattatgaaaataaaatctccgattacctcaatgcccgtcatattaaaggcaccttctatgtcaacggcaataactacgattgcatttacgatgaggcgatagtgaagcacttaaagcgcagtaagaatgttacctcttatcaaaaaaatatccccaccattgcctgcttatcatagattgttgttggcccaagctttctcccagggccacttgattggatcacatagttagtcatacctaccccctggtttccgcgagaggtctcttggctcattgattcttacaacaatccttcgatgttgcccggttacacaatctttgtttagcatggtcacatgcaaatatcagcagcttatcggccgcagaattaaaccaacagcttgacttagtcgaagtagcattaatcagttcgtctcattcaatttctgtcagactttttctccgagtaacatgcttagtattttgtgattttgtttggccatccagagattcttggagtcaagccgaaattcttccgacctccttatggtaagcttgacgaattatcaaaccagatgatgtgttgttaggatttttcgctgaagatatgagataattccgccaggtgcattcgaccagaagagcttggcgatattgaagaaacgggggtacatcgtggccaactggtcatttgactccgaagacgctgtgggagcgacgcccgagcaatcgatggcatcttataaggaactctcgaagcagtttccggcctctcagatcacactcaaccatgagacttaccaaactacggccgagaaagtgaccccatatgccgtgcctctgttgcaaaaggccggatataagctagtgcatatatccgaatgcttgggaacgggcaccaaattgactgatctctaccaatgggtcggaaagccttctgtcagagatgtaagttcaagttctgctcccccaacagttgaaaacatcttgaattgacttgtggccttggattcaggcatcgtggacgtgcaatgggaccccagctccaacggacaactaa

SEQ ID NO: 3 - protein, 247 aa, puccinia graminis
SPFTTRATEVIPDPATTCSRPGLAALTYDDGPYDYENKISDYLNARHIKGTFYVNGNNYDCIYDEAIVKHLKRTFSQGHLIGSHTWSHANISSLSAAELNQQLDLVEVALIKILGVKPKFFRPPYGAFDQKSLAILKKRGYIVANWSFDSEDAVGATPEQSMASYKELSKQFPASQITLNHETYQTTAEKVTPYAVPLLQKAGYKLVHISECLGTGTKLTDLYQWVGKPSVRDASWTCNGTPAPTDN

SEQ ID NO: 4 - DNA, 818 bp, unknown; identical to SEQ ID NO: 1
  schizosaccharomyces pombe and Puccinia graminis

(19) United States
(12) Patent Application Publication
(10) Pub. No.: US 2011/0087012 A1
(43) Pub. Date: Apr. 14, 2011

(54) NUCLEIC ACID MOLECULES CODING FOR A PROTEIN WITH DEACETYLASE ACTIVITY, SAID PROTEIN, AND METHOD FOR THE PRODUCTION OF CHITOSAN

(76) Inventors: Beate Kinga Jaszczuk, Munster (DE); Bruno Moerschbacher, Munster (DE); Andreas Schaff, Munster (DE)

(21) Appl. No.: 12/601,440

(22) PCT Filed: May 21, 2008
(86) PCT No.: PCT/EP08/04079

§ 371 Date: Sep. 28, 2010

(30) Foreign Application Priority Data
May 22, 2007 (DE) 10 2007 024 085.8

(51) Int. Cl. C08B 37/08; C07H 21/04; C12N 9/78; C12N 15/63; C12N 1/19; C12P 19/26; B82Y 5/00

(52) U.S. Cl. 536/20; 536/23.2; 435/227; 435/320.1; 435/254.2; 435/84; 977/773; 977/795; 977/906

(57) ABSTRACT

The invention relates to nucleic acid molecules selected from the group comprising: a) nucleic acid molecules that code for a form of the polypeptide with the derived amino acid sequence according to SEQ ID No. 3, said polypeptide having a deacetylase activity; b) nucleic acid molecules that comprise the nucleotide sequence according to SEQ ID No. 1 or SEQ ID No. 2 and that code for one form of said polypeptide; c) nucleic acid molecules that code for a fragment or a derivative of a polypeptide that is coded by a nucleic acid molecule according to a) or b), wherein one or more amino acid groups are conservatively substituted in said derivative as compared to said polypeptide, and wherein said fragment or derivative has a deacetylase activity; d) nucleic acid molecules that have a sequence identity of at least 95% with a nucleic acid molecule according to a) to c), and that code for a polypeptide with deacetylase activity; e) nucleic acid molecules that have a sequence identity of at least 70% with a nucleic acid molecule according to a) to c) and that code for a polypeptide with deacetylase activity; f) or the complementary string of a nucleic acid molecule according to a) to e).

### FIELD OF APPLICATION

This invention pertains to nucleic acid molecules according to the preamble of claim 1 coding for a protein with deacetylase activity, said protein and a method for the production of those.

### BACKGROUND OF THE INVENTION

Said protein with deacetylase activity is of particular interest for the production of Chitosan which, due to its special characteristics, has lately received increased attention.

Chitosan is a generic term for linear copolymers from β-1,4-glycosidically bonded n-acetyl glucosamine (GlcNAc) and glucosamine moieties (GlcN).

This is basically partially deacetylated chitin (poly-GlcNAc); for this reason there is no defined transition between chitin and chitosan. Usually, on speaks of chitosan starting at a degree of deacetylation (i.e. starting at GlcN content) of more than 40%. Due to the deacetylation, chitosan is soluble in organic acids while chitin is not.

One differentiates furthermore between low-molecular (M˜150,000 D), medium-molecular (M˜400,000), and high-molecular (M˜600,000 D) types of chitosan.

Chitosan is one of the few biopolymers which, under physiological conditions, carries a positive net charge. Due to this fact, chitosan interacts with anionic substances like proteins, DNA, fatty acids, and phospholipids. These physico-chemical interactions are made responsible for the different biological effects of chitosan.

Chitosan is very rarely found in nature; it is primarily found in the cell walls of fungi of the cygomycetes class.

Chitosan for commercial use is therefore produced from chitin. Chitin is a widespread biopolymer, and is found primarily in the cell walls of fungi as well as in the exoskeleton of most arthropodes, in particular, crustaceans. The large quantities of crustacean shells accrued in aqua-culture and in the crab-fishing industry are an economical source of chitin.

In the state of the art, chitosan is chemically manufactured from chitin. First, the crustacean shells are pulverized and afterward treated with an excess of HCl (0.25-1%, 12-24 hrs, room temperature) to remove the lime, which is 30% (w/w) of the shell material. The material is then washed, followed by a step to remove the protein using an excess of hot, watery NaOH (0.25-1 M, 1-3 hrs, 60-70° C.). The product is then washed, neutralized and dried.

The chitin recovered with this method then undergoes a partial deacetylation. For this purpose, the chitin is repeatedly treated with a hot concentrated liquor, preferably NaOH (35-50%, 1-3 hrs, 90-130° C.).

In some cases, this is followed by a decolorization step, e.g. with UV, O₃ or H₂O₂. The product is then washed, neutralized and dried.

The described process leads to products with a random acetylation pattern, and to a partial depolymerization of the polymer as well.

In addition, the product may exhibit indissoluble areas with a very high degree of acetylation, originating from the inner, crystalline areas of the chitin particles.

Chitosan produced in this manner is therefore very heterogeneous in regard to the degree of polymerization, degree of acetylation, and acetylation pattern. Nonetheless, this product is suitable for number of applications.

More sophisticated application, however, require chitosans of a higher quality, i.e. chitosans with a more precisely defined degree of polymerization (DP), degree of acetylation (DA), and acetylation pattern.

Such chitosans, if at all, can only be produced and cleaned up with great effort from the above described products.

Therefore, the approach has been pursued for some time now to produce chitosan from chitin using appropriate chitin deacetylases. It is suspected that, in particular, marine microorganisms feature deacetylases in order to break down exoskeletons of crustacean carcasses.

Tanaka et al. (J Biol Chem. 2004; 279: 30021-7) have described an diacetylchitobiose-deacetylase from the hyperthermophile archaebacterium thermococcus kodakaraensi.

The recombinant deacetylase indicated deacetylase activity toward n-acetylchitooligosaccharides; the deacetylating domain exhibits a specificity for the non-reducing GlcNAc-moiety.

The enzyme also exhibited deacetylase activity toward GlcNAc monomers. The derived amino acid sequence was attributed to the LmbE protein family, as which N-Acetylglucosaminyl-phosphatidylinositol-De-N-acetylases and 1-D-myo-inosityl-2-acetamido-2-deoxy-alpha-D-glucopyranoside-deacetylases are classified as well.

It turned out that thermococcus deacetylase is not suitable for the economically feasible production of chitosan from chitin. If at all, it can only be used for the production of some specific oligomers.

In addition, the expression rate of this protein is relatively poor, so that the sought-after protein can be produced in very small quantities only. Furthermore, the protein is most active at 75° C. and pH-value of 8.5. These conditions are for many applications not suitable. Another factor is that said protein deacetylizes only the first GlcNac position at the non-reducing end, and accepts a maximum of one pentamer as substrate, with hexamer or high polymer chitosan, however, there is no more deacetylase activity.

In summary, the only conceivable commercial application is therefore the deacetylation of the first, non-reducing position of oligomers with a maximum length of 5 units.

Blair et al. (Biochemistry, 2006, 45 (31), 9416-9426) have for the first time described the structure and the acting mechanism of a chitin deacetylase on the pathogenic fungus colletotrichum lindemuthianum (pathogen of Anthracnose).

This deacetylase also exhibits a number of drawbacks, which make it unsuitable or at least little suitable for industrial use. For example, this deacetylase can, at a maximum, deacetylize only every second sugar position. Therefore, the theoretically achievable maximum degree of deacetylation is around 50%. There is also the fact that this deacetylase does not allow deacetylation in blocks.

### DESCRIPTION OF THE INVENTION

Therefore, one of the objectives of the present invention is to provide a deacetylase enzyme with deacetylase activity toward GlcNAc molecules and polymers (chitin, chitosans), which can be cost-effectively manufactured in large quantities and which exhibits activity conditions, which make it more suitable for industrial processing.

Another objective of the present invention is to provide a method for the manufacturing of chitosan from chitin, which is more economical and environmentally friendlier than the method described in the beginning and/or the fabrication of more precisely defined chitosans.

Another objective of the present invention is to provide the amino acid sequence and the appropriate nucleic acid sequence for said deacetylase enzyme.

This objective is met with a nucleic acid molecule, a protein and a method according to the independent claims. The subclaims indicate preferred embodiments.

Accordingly, a nucleic acid molecule is provided, that is chosen out of a group consisting of:
a) Nucleic acid molecules coding for a form of the polypeptide with the derived amino acid sequence according to SEQ ID No: 3, whereby said polypeptide exhibits a deacetylase activity;
b) Nucleic acid molecules exhibiting the nucleotide sequence according to SEQ ID No: 1 or SEQ ID No: 2, and coding for a form of said polypeptide;
c) Nucleic acid molecules, coding for a fragment or a derivative of a polypeptide, which is coded by a nucleic acid molecule according to a) or b), whereby in said derivative one or multiple amino acid moieties in comparison to said polypeptide are conservatively substituted, and whereby said fragment or derivative has a deacetylase activity; Nucleic acid molecules exhibiting at least a 95% sequence identity with one nucleic acid molecule according to a)-b), and coding for a polypeptide with deacetylase activity;
e) exhibiting at least a 70% sequence identity with one nucleic acid molecule according to a)-b), and coding for a polypeptide with deacetylase activity;
f) or the complementary strand of a nucleic acid molecule according to a)-e).

In the following, the term “fragment” shall be understood as a polypeptide exhibiting at least one less amino acid than the originally utilized polypeptide.

In the following, the term “derivative” shall be understood to mean a polypeptide exhibiting at least one amino acid substitution in comparison to the originally used polypeptide.

Generally, however, the above definition also covers such nucleic acid molecules exhibiting a sequence deviating from the used sequences SEQ ID No: 1 or SEQ ID. No: 2, where these differences in sequence do not manifest themselves in sequence deviations of the coded polypeptide since it is a so-called “silent point mutations” (point mutations in the third location of a triplet) which, due to the degeneration of the genetic code do not lead to an amino acid substitution (so-called “wobble” mutations).

The term “conservatively substituted” shall in the following mean a substitution of a nucleic acid molecule, i.e. one or multiple base exchange mutations resulting in that an amino acid originally located at one point is substituted by an amino acid, which changes the properties of the polypeptide not at all or not significantly. As a rule, this applies to substituting amino acids with similar chemical properties.

Among others, the amino acids alanine, Glycine, Isoleucine, Leucine, Methionine and Valin are summed up as aliphatic amino acids. Phenylalanin, tryptophane and tyrosine count as aromatic amino acids; serine, threonine, asparagine, glutamine and tyrosine count polar amino acids; lysine, arginine and histidine count as alkaline amino acids, asparagine acid (aspartate) and glutamine acid (glutamate) count as acid amino acids, and cysteine and tyrosine count as amino acids with ionizable moieties.

The coded polypeptide with deacetylase activity, as the inventors surprisingly found, is a chitin deacetylase (CDA), in particular chitin deacetylase (CDA), in particular, chitin deacetylase from black wheat rust (Puccinia graminis).

Evidence for the existence of a chitin deacetylase resulted from experiments with axenic cultures of the fungus, which worldwide can only be performed in the laboratories of the inventors. Here it was first proven, that P. graminis modifies its cell wall at the moment of penetration into the plant from chitin to chitosan, and second, in zymograms with chitin substrate at least two (iso) forms of deacetylizing enzymes. The inventors were able to isolate the sequence of a putative polysaccharide deacetylase by screening a Lambda-cDNA bank. The primary identification was based on the detection of the polysacch_deac_{—}1 pattern stored in the PFAM database. The obtained information enabled the inventors to isolate the corresponding genomic sequence from the p. graminis genome, and to analyze the sequence in regard to its intron/exon structure.

Regarding the peptide sequence, the protein exhibits the already known sequence pattern of a typical polysaccharide deacetylase (PFAM: polysacch_deac_{—}1). This pattern extends nearly along the full length of the amino acid sequence but consists of only a few preserved positions and longer, intervening variable areas. N-terminal carries the protein in addition a secretion signal distinctly recognized by way of signal_P, which is why the inventors suspected an extra-cellular localization of the protein.

The inventors have determined that fungi of the type Puccinia graminis (scientifically Puccinia graminis f.sp. tritici; abbreviation: “Pgt”) partially deacetylize the amount entering their cell wall at the instant of infection.

The inventors suspect that the fungi disguise themselves in this manner in order to suppress the antimycotic immune response of the plant.

Thereupon, the inventors have for the first time described and isolated this deacetylase, verified its effect and recognized and described its suitability for the industrial production of chitosan.

In particular, the inventors suspect an outstanding suitability of such enzymes for the production of chitosans with non-random and non-blockwise acetylation pattern. Such chitosan products could be applied, among others, in medicine and in crop protection since its biological activity strongly depends on the named specific distribution patterns, which cannot be synthesized with conventional chemical methods.

Especially preferred is a nucleic acid molecule consisting of a secretionary signal sequence and said nucleic acid molecule coding for said deacetylase.

The mentioned signal sequence codes for secretionary signal peptide. Such signal peptide is a component of a protein, which is coded together with the sequence for the actual protein on the cDNA, and contains information about how and where this protein shall be transported inside the cell. Proteins designed for the transport into the endoplasmatic reticulum (ER), for example, and may therefore intended for subsequent secretion, have a signal in their amino acid sequence, which specifically guides them to the ER membrane. In secretoric proteins, the signal consists of an n-terminal localized amino sequence 15 to 40 amino acids long. Even though the primary structure of these signal peptides is not preserved,

three different areas can be differentiated: a central hydrophobic core is n-terminally flanked by positively charged amino acids and c-terminally by polar amino acids. In most cases, the signal peptide—after penetrating the membrane—is separated from the actual protein by the signal peptidase enzyme. In doing so, the interface is defined by small amino acid moieties in positions −3 and −1 of the c-terminal polar region of the signal sequence.

A typical eukaryotic signal peptide, which effects the transport of the protein to be synthesized into the ER, exhibits the following amino acid sequence, for example:

MMSFVSLLVGIFWATEAEQLTKCEVFQ

The sequence coding to the signal peptide—because it is located in the area of the 5′-end of the nucleic acid sequence—is synthesized first during the translation at the ribosome, so that, when the signal peptide protrudes far enough out of the ribosome, the SRP (“signal recognition particle”, a medium-sized riboprotein) attaches to the nascent polypeptide chain and the ribosome and stops the translation. At the surface of the (rough h) ER is an SRP receptor that binds the ribosome and positions it at the translocon. The SRP is then split off and can be used again as marker. The polypeptide chain is now further synthesized by the translocon into the lumen of the ER. Afterwards, the signal peptide enzyme removes the signal sequence. When the translation is finished, the protein in the ER lumen will be folded—with chaperons, if necessary. Further steps may be added, which are combined under the term of post-translational modification, which was introduced earlier.

In biotechnology, such signal peptides are used to ensure that the protein to be manufactured will be secreted into the medium surrounding the cells. For this purpose, an expression system is used, where a coding cDNA for the protein to be manufactured is cloned into a 3′ located region of a cDNA coding for a signal peptide. As a rule, this takes place via a restriction interface downstream from the signal sequence. Since this sequence usually exhibits at least two nucleotide triplets, the result is a construct where the signal sequence and the coding sequence for the protein to be created is separated by at least two nucleotide triplets; the translated protein therefore exhibits a minimum of two amino acid moieties between the signal peptide and the protein to be created. Accordingly, a nucleic acid molecule is provided, which codes for the signal peptide, and which is characterized in that the nucleic acid molecule in the area of its 3′-end exhibits an interface for a restriction endo-nuclease.

Native nucleic acid molecules coding for signal peptides, often do not exhibit any restriction interface, at least not in the region of their 3′-end. It is therefore preferably provided that the interface for a restriction endo-nuclease is generated by a silent point mutation in the native nucleotide sequence of the nucleic acid molecule for the signal peptide. In this manner, the function of the signal peptide is maintained, and the advantages mentioned above take effect.

According to the invention further provided is a method for the production of a protein with deacetylase activity, exhibiting the following steps:
a) if applicable, cloning of a nucleic acid molecule according to the above definition in an expression vector,
b) transfection of nucleic acid molecules and the expression vector into the expression system,
c) Incubation and/or fermentation of the expression system;
d) if applicable, specific induction of the protein expression;
e) Cleaning of the expressed protein with deacetylase activity.

The expressed protein is a chitin deacetylase of black wheat rust (Puccina graminis).

The term “cloning” shall in the following be understood to mean any techniques that allow the integration of nucleic acid molecules according to the above definition into an expression vector. In this respect, various techniques are well known to the professional, the most significant of which are described, for example, in the textbook “Molecular Biotechnology” by Michael Wink (1. Edition, (September 2004, Wiley-VCH), or in other textbooks and publications known to the professional.

The term “expression vector”, the use of which is optional, shall be understood by the professional to mean generally known transportation molecules for the transfer of a foreign nucleic acid molecule into a receptor cell. They include in particular plasmides, A-phages, cosmides, phasmides, P1-phage, BAC's, PAC's, and YAC's. Other suitable vectors are described in the textbook “Molecular Biotechnology” by Michael Wink (1. Edition, (September 2004, Wiley-VCH), for example, or in other known textbooks and publications.

The optionally used expression vector is preferably the plasmide pMel1, which is used in particular in Schizosaccharomyces pombe as the preferred expression system. This plasmide is shown in FIG. 3.

The term “transfection” shall in the following be understood to mean the introduction of a foreign nucleic acid molecule into a receptor cell, in particular with the assistance of an expression vector.

The terms “incubation” and/or “fermentation” of the expression system shall in the following be understood to mean the holding of the expression system under growth and cell-division-producing conditions. These conditions (in particular pO₂, pH, temperature, media composure, etc.) are known to the professional from the technical literature.

It is furthermore preferably provided that the expressed protein with deacetylase activity is cleaned up with affinity chromatography, gel filtration and/or ion-interchange chromatography. Here, in particular the combination with a His-tag will be considered. This is a tag consisting of multiple histidine moieties attached to the protein to be cleaned. The coding DNA is usually part of the expression vector and is translated together with the cDNA of the protein to be expressed.

The basic principle of this cleaning process is the so-called “IMAC” (Immobilized Metal Affinity Chromatography). In this process, two times positively charged ions like Cu²⁺, Ni²⁺, Co²⁺ or Zn²⁺ are immobilized by a chelizing ligand to a matrix and interact with the side chain of the histidine moieties of the His-tag.

Other affinity tags that can be used according to the invention are, for example, maltose-binding protein (MBP) or Glutathionyl-S-Transferase (GST), but also the S-tag, HAT-tag, calmoduline-binding peptide, chitin-binding peptide and some cellulose-binding domains. The utilized cleaning mechanisms are similar.

The invention also provides an expression vector that contains the above described nucleic acid molecule. Such expression vector is shown in FIG. 2, for example.

Also according to the invention is a host cell that was transformed with such nucleic acid molecule in such expression vector, whereby this cell functions as expression system. Such host cell may be an eukaryotic host cell. Preferred are yeast cells, in particular brewer's yeast (Saccharomyces cerevisiae) and fission yeast (Schizosaccharomyces pombe).

Generally, all eukaryotic expression systems (i.e. fungi, including yeast, unicellular organisms, plants, insect cells, and cells of mammals) are suitable for this purpose. Usually, functional proteins can be expressed and secreted only in combination with a secretion signal, that functions well within the respective system. It is therefore preferred to utilize the described vector in other types of yeast or to apply the principle. In this respect, we are thinking in particular of: Pichia pastoris, Hansenula polymorpha, Klyveromyces lactis.

Further provided by the invention is a polypeptide or protein, which is coded by the above described nucleic acid molecule or which can be produced with the above described process, whereby this polypeptide and/or protein exhibits deacetylase activity. This polypeptide or protein preferably exhibits an amino acid sequence that is shown in SEQ ID No 3.

In deviation, the polypeptide or protein may exhibit an amino acid sequence where one or several amino acid moieties are conservatively substituted. In respect to the term “conservatively substituted”, we refer to the explanation above.

This polypeptide or protein is a chitin deacetylase from black wheat rust (Puccinia graminis).

Furthermore provided is a process for the manufacture of chitosan from chitin, where N-acetyl-glucosamine moieties (GlcNAc) are deacetylated in a GlcNAc monomer or oligomer, a GlcNAc polymer, a copolymer from GlcNAc and glucosamine, or in a chitin molecule with such chitin deacetylase.

In this process, the preferred pH-value is a setting between 3 and 12 and a temperature between 5 and 80° C. Buffers are in particular tris, bicines, phosphates and borat. The preferred buffer concentration is 10-100 mM, especially preferred 40-60 mM. Also preferred, is the use of co-factors, e.g. Zn (1 mM).

In an especially preferred embodiment, the pH-value ranges between 6 and 11, and the most preferred pH-value is between 8 and 10. Especially preferred is a temperature range between 20 and 60° C., and most preferred a temperature between 30 and 50° C.

In order to optimize the process conditions as part of the experiment, the inventors digested chitohexamers an acetylation of 100% (DP6/DA100) and 42% (DP6/DA42) 27 hrs at 37° C. and different pH-values with the Pgt-CDA according to the invention. The products of the reaction as well as the control batches without enzyme were separated by DC and analyzed by fluorescamine assay. As a result, the highest deacetylation activity (from 100% to 64% and from 43% to 33%) was determined at a pH-value of 9.0; at pH 6.0 (from 100% to 84% and from 43% to 35%) and at a pH-value of 4.0 (from 100% to 99% and from 43% to 39%) the activity was significantly less.

Provided as an alternative is a process for the manufacture of chitosan, where n-acetyl-glucosamine monomers (GlcNAc) are deacetylated with such chitin deacetylase in order to create glucosamine monomers (GlcN), which in another substep are co-polymerized with untreated GlcNAc-monomers in order to obtain chitosan.

It may, however, also be provided that the chitin deacetylase according to the invention will be used for the acetylation of glucosamine moieties (GlcN) in a GlcN-mono or oligomer, a GlcN-polymer, a copolymer of GlcNAc and glucosamine or a chitosan molecule.

Through the selection of appropriate (i.e. usually unfavorable) reaction conditions (temperature, pH, reversal of the reaction equilibrium by withdrawal of acetylated substrate and introduction of deacetylated substrate) the CDA can be forced into a reversal of the direction of its reaction as is practically the case with all enzyme-catalyzed reactions. When this is done, the CDA can be used for the reacetylation of especially chitosan substrates. The resulting products will in the following be called “acetylized products”; they may, of course, also be chitosans with different acetylation patterns and content but, in particular, as chitin.

This application is of great general interest since it may again lead to products with defined acetylation patterns and acetylation content.

Furthermore provided by the invention is a chitosan or an acetylized product as just described, which can be manufactured with one of the described methods. Such chitosan or acetylized product is characterized by a polymerization and acetylation that can be precisely defined as well as a precisely definable acetylation pattern.

Finally, according to the invention, the use of such chitosan or acetylated product is provided for at one of the applications, selected from the group comprising:
a) Immobilizing of enzymes and cells in biotechnology processes;
b) Strengthening and/or coloring of human and/or animal hair;
c) Skin and/or hair cosmetics;
d) Protection and preservation of seeds and/or grain;
e) Adsorption of endotoxins and nucleic acid
f) Paper processing.

Other applications according to the invention include:
a) Polymer carriers for catalysts;
b) Intermediate product for the synthesis of fine chemicals;
c) Additive whey feed;
d) Composting accelerator;
e) Packaging material;
f) Clearing agent for juices;
g) Flocculation agent;
h) Dietetic fiber for foodstuffs;
i) Coating agent for foodstuffs;
j) Wound bandages;
k) Surgical, suture material;
l) Dietetic food additive;
m) Contact lenses;
n) Loudspeaker membrane;
o) Embedding agent for electron microscopy, or
p) Precipitating agent for drinking water and sewage treatment;
q) Matrices for tissue engineering;
r) Chitosan particles for drug, gene and vaccine delivery

Many applications benefit from the precisely definable degree of acetylation and the precisely definable acetylation pattern of the chitosans manufactured pursuant to the invention, which manifests itself in particular in a defined solubility and a defined net charge of the respective polymer. Both parameters are instrumental for the listed applications.

### FIGURES AND SAMPLES

The invention will in the following be explained in greater detail based on the figures and samples that will be discussed. Please note that the figures are only descriptive, and are not intended to restrict the invention in any form or fashion.

The terms chitohexaose and chitohexose will be used interchangeably in the following samples.

FIG. 1 shows a screening the culture medium, in which transformed Schizosaccharomyces pombe cells were cultivated with a construct (pMel-CDA). The cells were cultivated in 10 ml cultures (Falcon tubes). The culture medium was analyzed 20 hours after induction. The cleaning of the Pgt-CDA from the culture medium according to the invention was performed with NiNTA-IMAC affinity chromatography.

Shown are in FIG. 1 a) PAGE gels of the most important cleaning steps stained with silver; FIG. 1b) a silver gel of the main elution fractions, and FIG. 1c) a western blot of the main elution fractions (anti-His tag). The gel paths are populated as follows:
R: Raw fraction (concentrated nutrient solution)
FT: Flow-Through fraction
W: Wash fraction
E8-E12: Elution fractions

The total volumes of the fractions were:
R: 100 ml
FT: 100 ml
W: 50 ml
E8-12: 1 ml

The path were populated with a 15 μl sample each.

FIG. 2 shows the results of the activity test of recombinant Pgt-CDA in comparison to chitosan Substrates. FIG. 2a) shows a zymogram (substrate: glycol-chitin, incubation: 37° C. in 50 mM BisTris, pH6 over night, negative stain with calcofluor after depolymerization of chitosan with nitrous acid).

FIG. 2b) shows the results of a thin-film chromatography (DC). In this case, the chitohexamers were digested with an acetylation of 100% (DP6/DA100) and 42% (DP6/DA42) 27 hours at 37° C. under two different buffer conditions (50 mM BisTris, pH 6.0 and 50 mM borat, pH 9.0) with recombinant Pgt-CDA.

The reaction products and the control batches without enzyme were separated with DC and then visualized.

The marking “- - -” indicates a batch without enzyme, the marking “CDA” indicates a batch with enzyme.

FIG. 3 shows the structure formula of a section from a copolymer of n-acetyl-glucosamine (GlcNAc; left) and glucosamine (GlcN; right), β-1,4-glycosidically linked. This section stands ideal-typically for the structure of chitosan.

FIG. 4 shows the reaction scheme of the deacetylation of glucosamine (FIG. 4A) by chemical method and with the help of the inventive chitin deacetylase (FIG. 4B). Analogous, the reaction schemes also apply to the deacetylation of n-acetyl-glucosamine moieties in a chitin or chitosan molecule.

The use of a chitin deacetylase according to the invention allows in contrast to the classic chemical deacetylation the creation of better defined and reproducible chitosans.

The also described utilization of the CDA according to the invention for the reacetylation is indicated in FIG. 4 by the broken-line reaction arrow.

FIG. 5 shows the mass-spectrometric analysis of the reaction products. 25 μg chitohexaose were incubated 24 hrs at 40° C. with 0.5 μg Pgt-CDA in reaction buffer (50 mM BisTris, pH 6.9; 100 mM NaCl; 1 mM ZnCl). The reaction products as well as the educt, which was incubated in the same manner but without enzyme, were analyzed with Maldi-TOF-MS (Bruker Daltonik Reflex 4).

Shown are the mass spectrum of the educt (black, top), and of the product (bottom, gray).

FIG. 6 shows the characterization and the kinetics of Pgt-CDA. FIG. 6a) shows the determination of the optimum pH-value: 27 nM Pgt-CDA were incubated at different pH-values in different buffers with 0.88 mM chitohexose as substrate at 37° C. for 19 hrs, following by the measuring if the Pgt-CDA activity. For pH-values in the range of 5.8-7.2, Bis Tris buffers were used, for the range from pH 7.4-9.4 bicine buffer was used. All buffers had a concentration of 25 mM. FIGS. 6b+d) show the effect of the incubation temperature on the activity: 74 nM Pgt-CDA was incubated with 0.88 mM chitohexaose in 50 mM Bis Tris pH 6.9 1 mM ZnCl₂ at different temperatures in a water bath. At the indicated times, samples were taken, inactivated with heat treatment and stored in closed condition at room temperature.

FIG. 6c) shows the effect of metal ions on the Pgt-CDA activity: 106 nM Pgt-CDA were incubated in 50 mM bicine buffer (pH 8.0) at 37° C. for 2 hrs with 0.22 mM chitohexose and different metal ions in form of their chloride salts at a concentration of 1 mM. As control, 50 mM bicine buffer without metal ions was used. FIG. 6e) shows Michaelis-Menten kinetics: 76 nM Pgt-CDA was incubated in 50 mM BisTris pH 6.9 ImM ZnCl₂ at 37° C. for 24 hrs with different substrate concentrations (S) of mit chitohexaose. KM and vₘₐₓ were determined by non-linear regression using the GraphPad Prism software.

In all assays, the relative or absolute activity was determined by fluorescamine marking of the obtained GlcN and subsequent fluorometry. The values show a median value of three (6a+C) or two (6b,d+e) independent reactions.

FIG. 7 shows a mass-spectrometric analysis of recombinant Pgt-CDA. Cleaned, recombinant pgt-CDA was analyzed pursuant to Naumann et al (2005). Peptides that were found and their positions in the native protein are shown in this figure.

FIG. 8 shows the enzyme deacetylation of chitosan substrates. Five different chitosans were deacetylated with Pgt-CDA. The starting substrates were (black bars from left to right): DP737/DA11; DP818/DA27; DP713/DA35; DP1442/DA56; DP3726/DA66. The substrates were incubated 24 hrs at 37° C. in 50 mM BisTris pH 6.9, 1 mM ZnCl with 20 nM Pgt-CDA. The amount of released acetate was determined for each reaction using a commercial acetate assay (R-Biopharm, Darmstadt) and the resulting degree of acetylation (DA) was measured (white bars).

FIG. 9 shows the salt tolerance of Pgt-CDA. Chitosan (DP3726/DA66—black bars) was incubated with 80 nM Pgt-CDA in 50 mM BisTris, pH 6.9, 1 mM ZnCl and variable salt concentrations at 40° C. for 19 hrs. The amount of released acetate was determined for each reaction using a commercial acetate assay (R-Biopharm, Darmstadt) and the resulting degree of acetylation (DA) was measured (white bars).

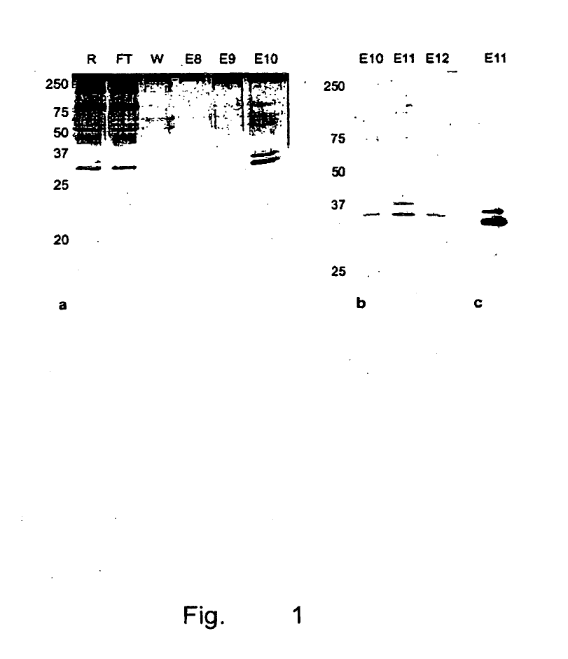

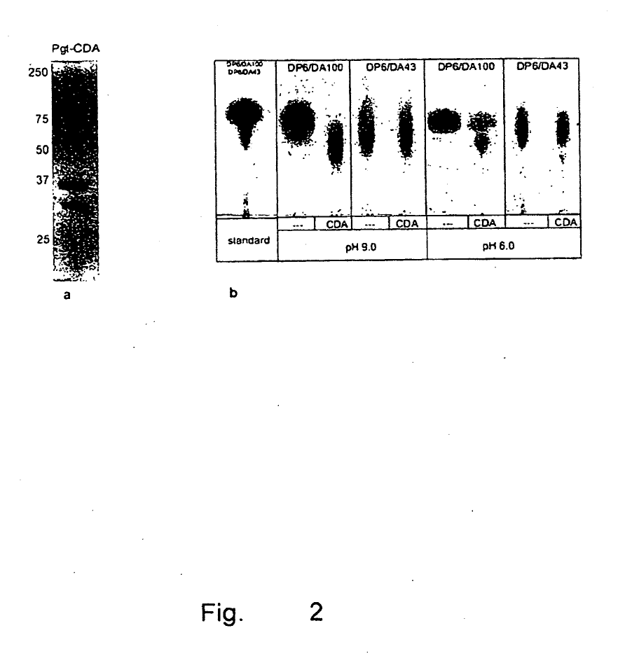

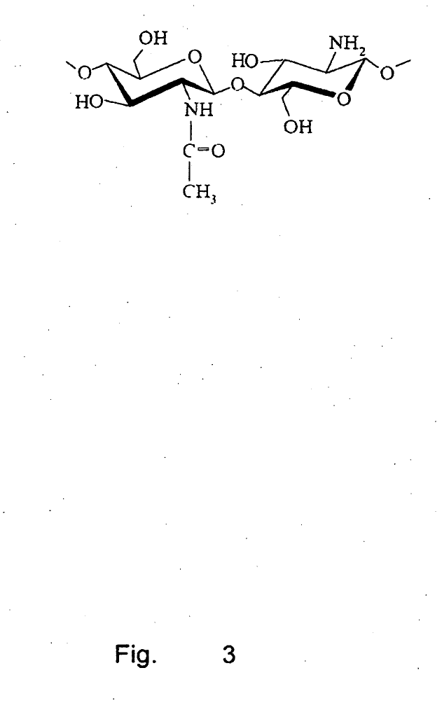

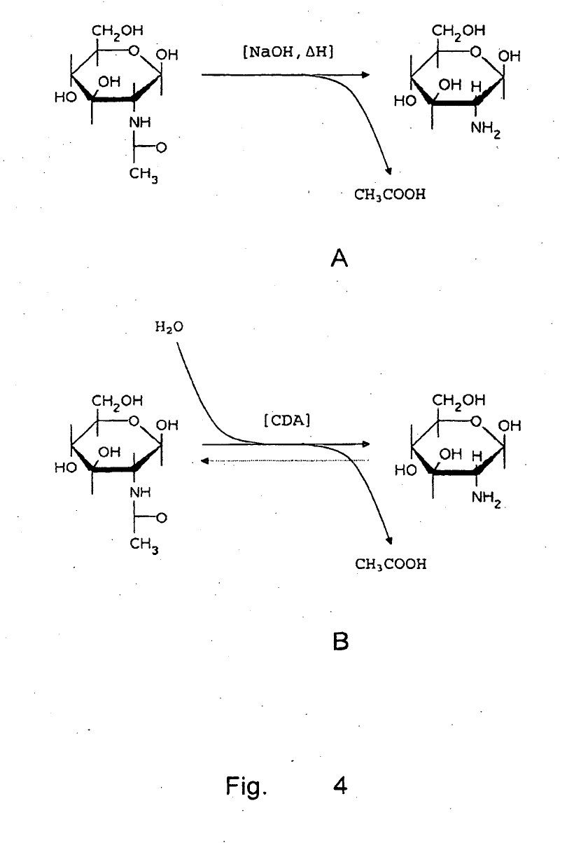

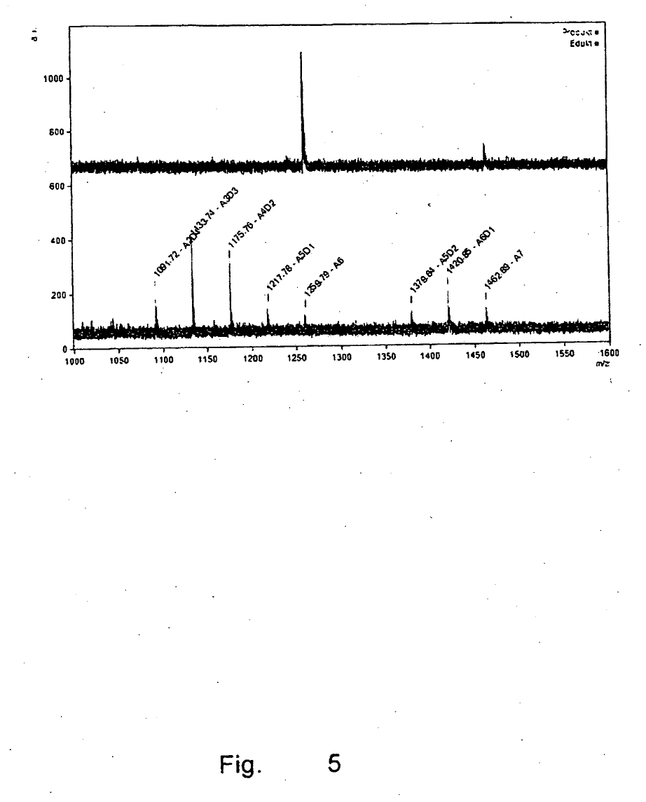

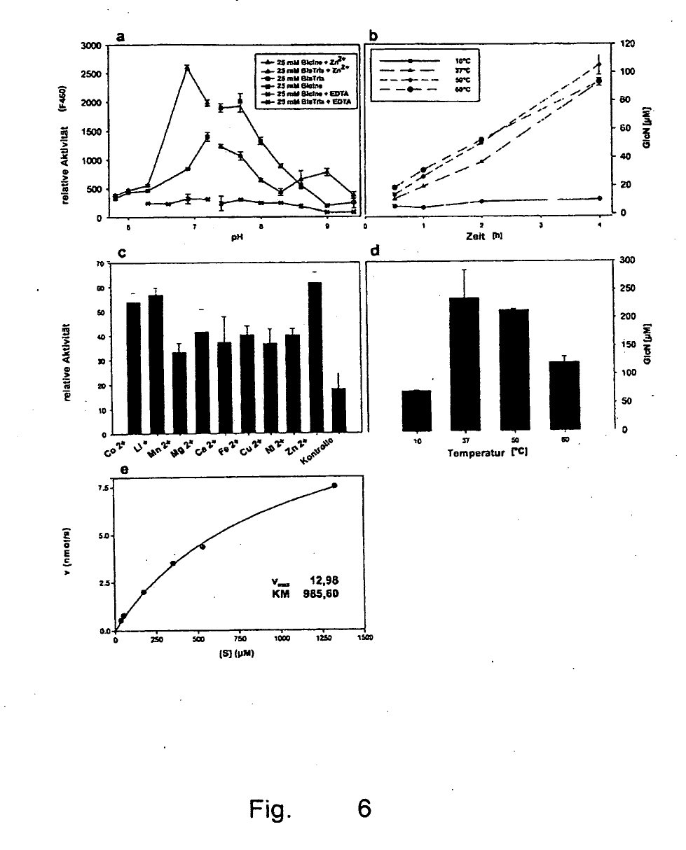

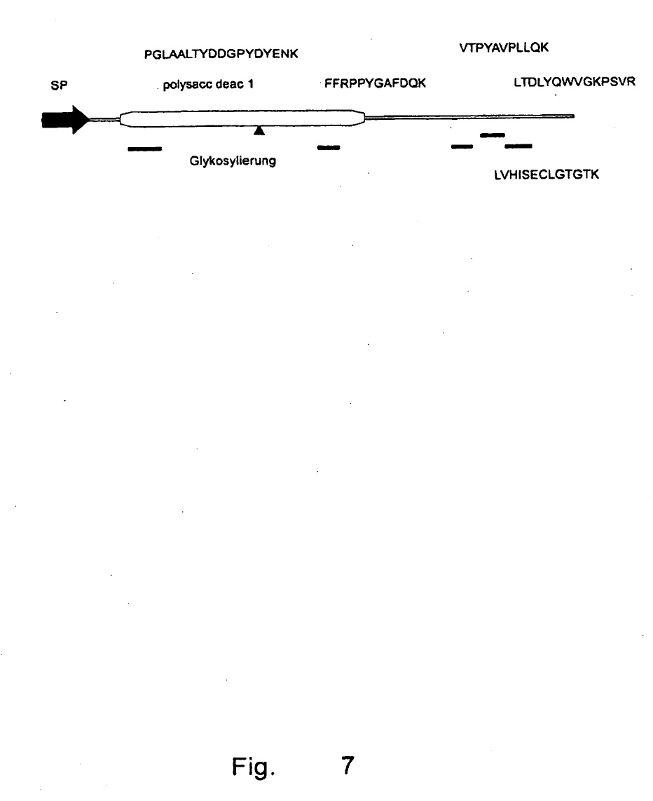

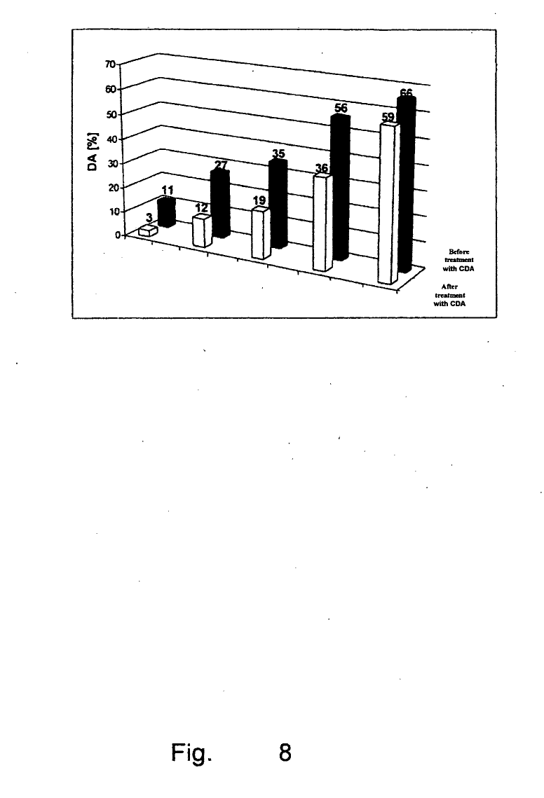

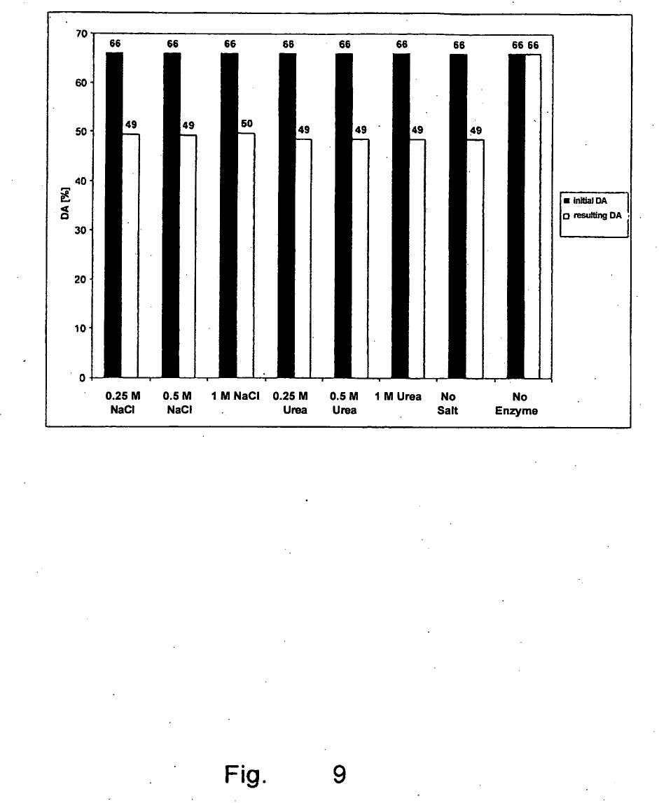

In summary, the experiments demonstrate a high stability of the enzyme under broadly variable abiotic conditions. Extreme salt tolerance combined with high thermal stability and a broad pH spectrum should enable the development of a one or two-stage process for the deacetylation of crystalline chitin.

1. Nucleic acid molecule selected from the group consisting of:
a) Nucleic acid molecules that code for a form of the polypeptide with the derived amino acid sequence according to SEQ ID No. 3, said polypeptide having a deacetylase activity;
b) Nucleic acid molecules that comprise the nucleotide sequence according to SEQ ID No. 1 or SEQ ID No. 2 and that code for one form of said polypeptide;
c) Nucleic acid molecules that code for a fragment or a derivative of a polypeptide that is coded by a nucleic acid molecule according to a) or b), wherein one or more amino acid groups are conservatively substituted in said derivative as compared to said polypeptide, and wherein said fragment or derivative has a deacetylase activity;
d) Nucleic acid molecules that have a sequence identity of at least 95% with a nucleic acid molecule according to a) to c), and that code for a polypeptide with deacetylase activity;
e) Nucleic acid molecules that have a sequence identity of at least 70% with a nucleic acid molecule according to a) to c) and that code for a polypeptide with deacetylase activity; or
f) The complementary string of a nucleic acid molecule according to a) to e).

2. Nucleic acid molecule according to claim 1, wherein the coded polypeptide with deacetylase activity is a chitin-deacetylase (CDA).

3. Nucleic acid molecule according to claim 2, wherein the coded polypeptide is a chitin-deacetylase of black wheat rust (Puccinia graminis).

4. Nucleic acid molecule, characterized in that said molecule consists of a secretoric signal sequence and a nucleic acid molecule according to the previous claims.

5. Method for the production of a protein with deacetylase activity consisting of the following steps:
a) possible cloning of a nucleic acid molecule according to the above definition into a expression vector;
b) transfection of nucleic acid molecules and the expression vector in an expression system,
c) incubation and/or fermentation of the expression system;
d) possible specific induction of the protein expression;
e) cleaning of the expressed protein with deacetylase activity.

6. Method according to claim 5, with the expressed protein being a chitin deacetylase from black wheat rust (Puccinia graminis).

7. Method according to one of claims 5-6, with the expression vector being the plasmide pMel1.

8. Method according to one of claims 5-7, with the expression system being Schizosaccharomyces pombe.

9. Method according to one of claims 5-8, wherein the expressed protein is cleaned with deacetylase activity via affinity chromatography and/or gel filtration.

10. Expression vector containing the nucleic acid molecule according to one of claims 1-4.

11. Host cell transformed with a nucleic acid molecule according to claim 1-4 or with the expression vector according to claim 10, wherein this cell functions as expression system.

12. Polypeptide or protein coded by the nucleic acid molecule according to claims 1 to 4, exhibits an amino acid sequence according to claim 1, or can be produced with the method according to claims 5-8, wherein this protein exhibits deacetylase activity.

13. Polypeptide or protein according to claim 12, wherein this protein is a chitin deacetylase from black wheat rust (Puccinia graminis).

14. Method for the production of chitosan from chitin, in which n-acetyl glucosamine moieties (GlcNAc) are deacetylated in a GlcNAc-Mono or oligomer, a GlcNAc polymer, a copolymer of GlcNAc and glucosamine or in a chitin molecule with a protein according to claims 12-13.

15. Method for the production of chitosan, in which n-acetyl glucosamine monomers (GlcNAc) are deacetylated with a Protein according to claims 12-13, in order to produce glucosamine monomers (GlcN), which are then, for example, copolymerized with untreated GlcNAc monomers to obtain chitosan,

16. Method for the acetylation of glucosamine moieties (GlcN) in a GlcN mono- or oligomer, a GlcN polymer, a copolymer of GlcNAc and glucosamine or a chitosan molecule characterized in that a protein according to claims 12-13 will be used.

17. Chitosan or acetylated product, producible by a method according to claims 14-16.

18. Use of a chitosan or acetylated product according to claim 17 for at least one of the applications selected from the group consisting of:
a) Immobilizing of enzymes and cells in biotechnology processes;
b) Strengthening and/or coloring of human and/or animal hair;
c) Skin and/or hair cosmetics;
d) Protection and preservation of seeds and/or grain;
e) Adsorption of endotoxins and nucleic acid
f) Paper processing.

19. Use of a chitosan or acetylated product according to claim 17 for at least one of the applications selected from the group consisting of:
a) Polymer carriers for catalysts;
b) Intermediate product for the synthesis of fine chemicals;
c) Additive whey feed;
d) Composting accelerator;
e) Packaging material;
f) Clearing agent for juices;
g) Flocculation agent;
h) Dietetic fiber for foodstuffs;
i) Coating agent for foodstuffs
j) Wound bandages;
k) Surgical suture material;
l) Dietetic food additive;
m) Contact lenses;
n) Loudspeaker membrane;
o) Embedding agent for electron microscopy, or
p) Precipitating agent for drinking water and sewage treatment;
q) Matrices for tissue engineering;
r) Chitosan particles for drug, gene and vaccine delivery
